(19) United States
(12) Patent Application Publication
(10) Pub. No.: US 2019/0083061 A1
(43) Pub. Date: Mar. 21, 2019

(54) COMPACT CALIBRATION FOR MECHANICAL THREE-DIMENSIONAL ULTRASOUND PROBE

(71) Applicant: Verathon Inc., Bothell, WA (US)

(72) Inventor: Joon Hwan Choi, Bothell, WA (US)

(21) Appl. No.: 16/131,307

(22) Filed: Sep. 14, 2018

Related U.S. Application Data
(60) Provisional application No. 62/559,791, filed on Sep. 18, 2017.

(51) Int. Cl. A61B 8/00; A61B 8/08; A61B 6/00

(52) U.S. Cl. CPC A61B 8/42; A61B 8/483; A61B 8/085; A61B 8/58; A61B 6/5258

(57) ABSTRACT

Systems and methods described herein allow for compact calibration of three-dimensional (3D) ultrasound probes. In one embodiment, a calibration device for an ultrasound probe has an open end to receive a nose portion of the ultrasound probe; a closed end including an inner surface; and a target secured to the inner surface, the target includes an echo-absorbing or echo-reflective material with different acoustic properties than the inner surface. The calibration device has an inner width dimension that is no more than two times the maximum nose diameter of the ultrasound probe.

### CROSS-REFERENCE TO RELATED APPLICATION

This application claims priority under 35 U.S.C. § 119, based on U.S. Provisional Patent Application No. 62/559,791 filed Sep. 18, 2017, the disclosure of which is hereby incorporated by reference.

### BACKGROUND OF THE INVENTION

Ultrasound scanners are typically used to identify a target organ or other structures in the body and/or determine features associated with the target organ/structure, such as the size of the organ/structure or the volume of fluid in the organ. An ultrasound probe typically includes one or more ultrasound transducer elements that transmit ultrasound energy and receive acoustic reflections or echoes from internal structures/tissue within a body. These reflections or echoes may be converted into three-dimensional (3D) data. Errors in the probe mechanism, such as small mechanical assembly deviations, can distort the 3D ultrasound data. The distortion can adversely affect measurement of features associated with the target organ/structure.

An external fixture is typically used for calibrating a single-element 3D ultrasound probe. The external fixture usually includes a large water tank with an ultrasound target. The external fixture is generally used on an annual basis, and the large size makes storage of the fixture inconvenient.

### BRIEF DESCRIPTION OF THE DRAWINGS

FIG. 1A is a schematic of a scanning system in which systems and methods described herein may be implemented;

FIG. 1B is a schematic of a scanning station including a compact calibration unit, according to an implementation described herein;

FIG. 2 is a schematic of a portion of an ultrasound probe of FIGS. 1A and 1B in an exemplary implementation;

FIGS. 3A and 3B are top and side views, respectively, of a calibration cup according to an implementation described herein;

FIGS. 4A and 4B are partial cut-away views of an ultrasound probe within the calibration cup of FIGS. 3A and 3B;

FIGS. 5A and 5B are a side view and a cut-away view of a portion of an ultrasound probe with a calibration cap attached, according to an implementation described herein;

FIGS. 6A and 6B are top and side cross-sectional views, respectively, of the calibration cap of FIG. 5;

FIG. 7 is a process flow diagram of a process for identifying calibration errors with compact calibration devices, according to an implementation described herein;

FIGS. 8A-8C are simplified diagrams illustrating phi offset detection for a probe with no error, according to an implementation described herein;

FIGS. 9A-9C are simplified diagrams illustrating phi offset detection for a probe with error, according to an implementation described herein;

FIGS. 10A-10C are simplified diagrams illustrating skew error detection for a probe with no error, according to an implementation described herein;

FIG. 11A-11E are simplified diagrams illustrating theta motion error detection for a probe with no error, according to an implementation described herein;

FIGS. 12A-12C are schematics illustrating additional target patterns for the calibration cap of FIG. 6A;

FIG. 13 is a partial cutaway view of an ultrasound probe equipped for accelerometer-based calibration, according to an implementation described herein;

FIG. 14 is a schematic of a portion of an ultrasound probe with corresponding gravity intensity profiles for different transducer orientations;

FIG. 15 is a process flow diagram of a process for estimating phi angle probe error using accelerometer data, according to an implementation described herein;

FIG. 16 is a schematic of a portion of an ultrasound probe showing vectors used for a phi motion integrity check;

FIG. 17 is a process flow diagram of a process for detecting phi motion probe error using accelerometer data, according to an implementation described herein;

FIG. 18 is a process flow diagram of a process for estimating theta motion error in probe using accelerometer data, according to an implementation described herein;

FIG. 19 is a process flow diagram of a process for estimating errors in an ultrasound probe using gravity angle information;

FIG. 20 is a block diagram illustrating exemplary physical components of the base unit of FIGS. 1A and 1B; and

FIG. 21 is a schematic of a portion of the ultrasound probe of FIGS. 1A and 1B according to another implementation.

### DETAILED DESCRIPTION OF THE PREFERRED EMBODIMENTS

The following detailed description refers to the accompanying drawings. The same reference numbers in different drawings may identify the same or similar elements.

Implementations described herein relate to compact calibration fixtures for identification of errors in data from ultrasound probes. Errors in the probe mechanism, such as mechanical alignment errors during assembly, can cause distortion of 3D ultrasound data collected by the probe. This distortion can negatively affect measurements of scanned organs (e.g., bladder volume, aorta diameter, prostate width/height, etc.). One common way to check for distortion requires use of external calibration fixtures, which usually include a bulky water tank with an ultrasound target. The size (typically a 20-by-30 centimeter footprint) and inconvenience of the external calibration fixtures typically precludes storage within the ultrasound station. Furthermore, these conventional external fixtures may be misplaced or lost between the infrequent calibration intervals.

According to implementations described herein, compact calibration fixtures are provided for an ultrasound probe. The calibration fixtures may be small enough to be stored in a mobile ultrasound station. In some cases, the calibration fixtures have a footprint just slightly larger than a dome diameter of the ultrasound probe, and in other cases the calibration fixture footprint may be equal to or smaller than the dome diameter. The calibration fixtures include an inner surface with a known target profile disposed thereon. In one implementation, the calibration fixture may be in the form of a cup or bowl into which the probe is inserted. In another implementation, the calibration fixture may be in the form of a cap that may be removeably attached to an end of the probe. The calibration fixtures may be re-usable or disposable. In one implementation, the calibration fixture may be incorporated into a cart for the ultrasound scanning system.

According to another implementation, probe calibration may be assisted or performed using feedback from a sensor (e.g., an accelerometer) mounted on a transducer assembly of the probe. As described further herein, in one implementation, the accelerometer can detect the direction of gravity, which can be used to measure the relative angle of the transducer from the gravity line. In another implementation, a three-axis accelerometer can be used to measure the relative angle between any two scanlines.

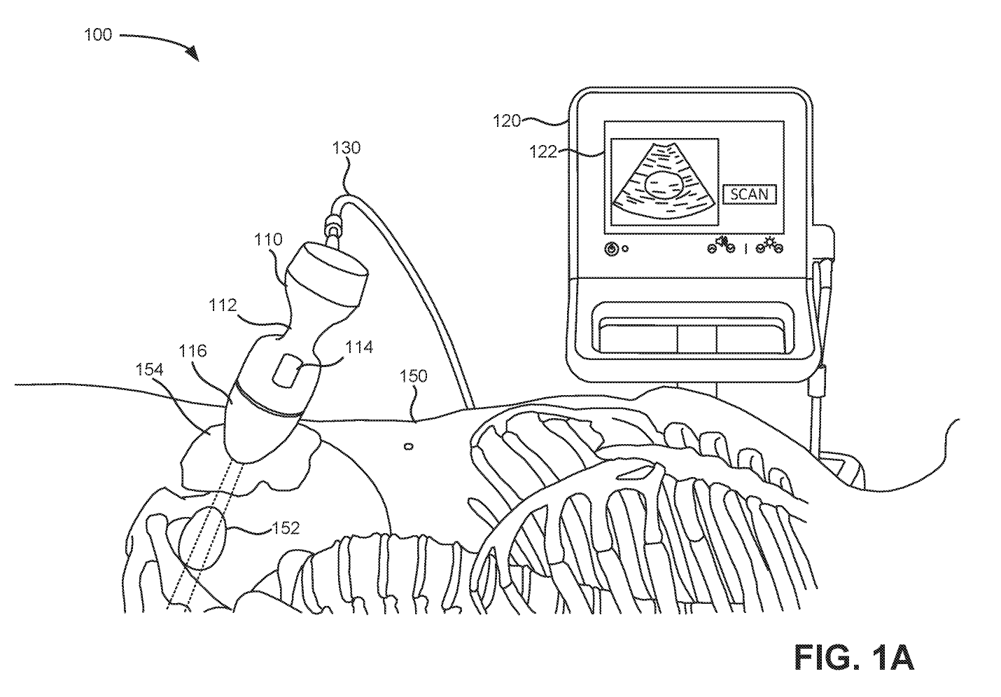

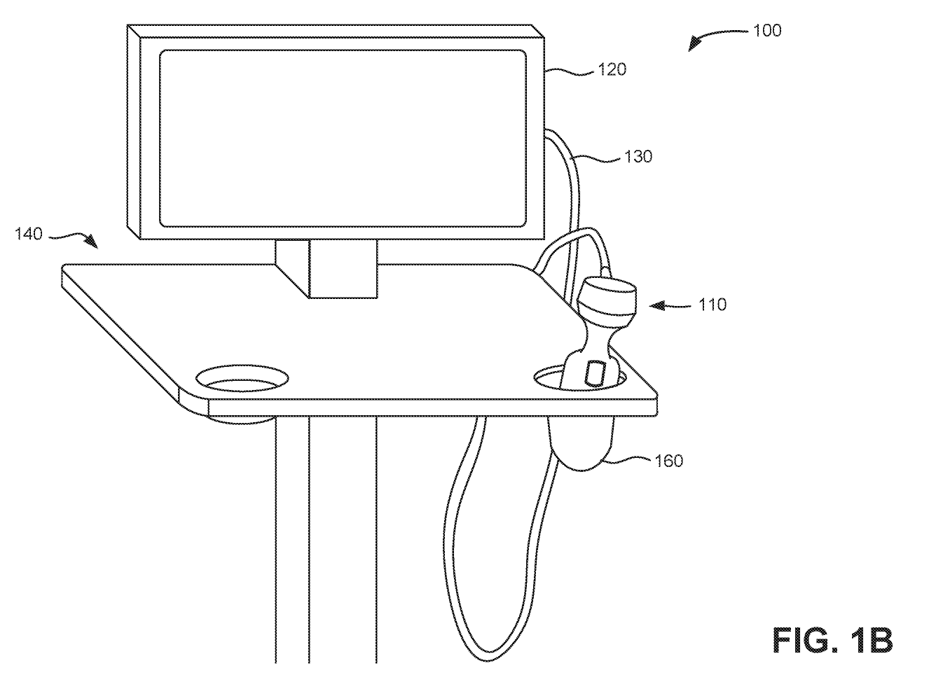

FIGS. 1A and 1B are schematics of a scanning system 100 in which systems and methods described herein may be implemented. Referring collectively to FIGS. 1A and 1B, scanning system 100 includes a probe 110, a base unit 120, and a cable 130.

As shown in FIG. 1A, probe 110 includes handle portion 112 (also referred to as handle 112), trigger 114 and nose portion 116 (also referred to as dome or dome portion). Medical personnel may hold probe 110 via handle 112 and press trigger 114 to activate one or more ultrasound transceivers and transducers located in nose portion 116 to transmit ultrasound signals toward the target organ of interest. In the example of FIG. 1A, probe 110 is located on pelvic area of patient 150 and over a target organ of interest, which in this example is the patient's bladder 152.

Handle 112 allows a user to move probe 110 relative to patient 150. Trigger 114 initiates an ultrasound scan of a selected anatomical portion while nose portion 116 is in contact with a surface portion of the patient when the selected anatomical portion is scanned. Nose portion 116 is typically formed of a material that provides an appropriate acoustical impedance match to the anatomical portion and/or permits ultrasound energy to be properly focused as it is projected into the anatomical portion. For example, an acoustic gel or gel pads, illustrated at area 154 in FIG. 1A, may be applied to patient's skin over the region of interest (ROI) to provide an acoustical impedance match when the nose portion is placed against the skin.

Probe 110 may communicate with base unit 120 via a wired connection, such as via cable 130. In other implementations, probe 110 may communicate with base unit 120 via a wireless connection (e.g., Bluetooth, WiFi, etc.). In each case, base unit 120 includes display 122 to allow a user to view processed results from an ultrasound scan, and/or to allow operational interaction with respect to the user during operation of probe 110. For example, display 122 may include an output display/screen, such as a liquid crystal display (LCD), light emitting diode (LED) based display, or other type of display that provides text and/or image data to a user. For example, display 122 may provide instructions for positioning probe 110 relative to the selected anatomical portion of a patient. Display 122 may also display two-dimensional or three-dimensional images of the selected anatomical region. In some implementations, display 122 may include a graphical user interface (GUI) that allows the user to select various features associated with an ultrasound scan.

To scan a selected anatomical portion of a patient, nose portion 116 may be positioned against a surface portion of the patient that is proximate to the anatomical portion to be scanned. The user actuates the transceiver by depressing trigger 114. In response, the transducer elements optionally position the transceiver, which transmits ultrasound signals into the body, and receives corresponding return echo signals that may be at least partially processed by the transceiver to generate an ultrasound image of the selected anatomical portion. In a particular embodiment, system 100 transmits ultrasound signals in a range that extends from approximately about two megahertz (MHz) to approximately 10 or more MHz (e.g., 18 MHz).

In one embodiment, probe 110 may be coupled to a base unit 120 that is configured to generate ultrasound energy at a predetermined frequency and/or pulse repetition rate and to transfer the ultrasound energy to the transceiver. Base unit 120 also includes one or more processors or processing logic configured to process reflected ultrasound energy that is received by the transceiver to produce an image of the scanned anatomical region.

In still another particular embodiment, probe 110 may be a self-contained device that includes a microprocessor positioned within the probe 110 and software associated with the microprocessor to operably control the transceiver, and to process the reflected ultrasound energy to generate the ultrasound image. Accordingly, a display on probe 110 may be used to display the generated image and/or to view other information associated with the operation of the transceiver. For example, the information may include alphanumeric data that indicates a preferred position of the transceiver prior to performing a series of scans. In other implementations, the transceiver may be coupled to a general-purpose computer, such as a laptop or a desktop computer that includes software that at least partially controls the operation of the transceiver, and also includes software to process information transferred from the transceiver so that an image of the scanned anatomical region may be generated.

As described above, probe 110 may include a transceiver that produces ultrasound signals, receives echoes from the transmitted signals and generates B-mode image data based on the received echoes. In an exemplary implementation, base unit 120 obtains data associated with multiple scan planes corresponding to the region of interest in patient 150. For example, probe 110 may receive echo data that is processed by base unit 120 to generate two-dimensional (2D) B-mode image data to determine bladder size and/or volume. In other implementations, probe 110 may receive echo data that is processed to generate three-dimensional (3D) image data that can be used to determine bladder size and/or volume.

As shown in FIG. 1B, scanning system 100 may include a cart 140 to provide convenient access and storage for probe 110, base unit 120, cable 130, and other accessories (not shown). Cart 140 may include a holding cup 160 to receive probe 110, as shown in FIG. 1B. Probe 110 may be placed in a holding cup 160 when not in use. According to one implementation, as described further herein, holding cup 160 may be configured to service as a calibration fixture for probe 110.

FIG. 2 is a schematic of an internal portion of probe 110 in an exemplary implementation. In the example of FIG. 2, probe 110 is configured to obtain 3D image data. Generally, probe 110 includes one or more ultrasound transceiver elements and one or more transducer elements within nose portion 116 that transmit ultrasound energy outwardly from nose portion 116, and receive acoustic reflections or echoes from internal structures/tissue within an anatomical portion. Referring to FIG. 2, probe 110 includes a transducer 210 connected to a base 260. The elements illustrated in FIG. 2 may be included within nose portion 116 of probe 110.

Transducer 210 may transmit ultrasound signals from probe 110 through a wall 220 of nose portion 116, indicated by reference 225 in FIG. 2. Transducer 210 may be mounted to a transducer bucket 215, which in turn is mounted to base 260 to allow transducer 210 to rotate about two perpendicular axes. A motor 230 (also referred to as theta motor 230) may be included to move a first axis or spine 240, and another motor 235 (also referred to as phi motor 235) may be included to move a second axis or shaft 250. For example, transducer 210 may rotate around first axis 240 with respect to base 260 and rotate around a second axis 250 with respect to base 260. The first axis 240, extending in a generally longitudinal direction of probe 110, is referred to herein as the theta (θ) axis. The second axis 250, extending in a direction orthogonal to first axis 240, is referred to herein as the phi (ϕ) axis. In an exemplary implementation, the range of theta and phi motion may be less than 180 degrees. In one implementation, the scanning may be interlaced with respect to the theta motion and phi motion. For example, movement of transducer 210 may occur in the theta direction followed by movement in the phi direction. This enables probe 110 to obtain smooth continuous volume scanning as well as improving the rate at which the scan data is obtained. Rotation of transducer 210 about axis 250 in the phi (ϕ) direction generates a scan plane 255

While a single transducer is shown in the implementation of FIG. 2, different configurations for probe 110 may be used. For example, as shown in FIG. 21 and described further below, the one or more ultrasound transducer elements may include a one-dimensional, a two-dimensional, or an annular array of piezoelectric elements that may be moved within nose portion 116 by a motor to provide different scan directions with respect to the transmission of ultrasound signals by the transceiver elements. Alternatively, the transducer elements may be stationary with respect to probe 110 so that the selected anatomical region may be scanned by selectively energizing the elements in the array.

Production/mechanical alignment errors in the manufacture of probe 110, knocks or dropping probe 110 during use of probe 110, or general wear of motors and other components of probe 110 can result in various types of calibration errors. Thus, routine calibration tests for probe 110 are recommended.

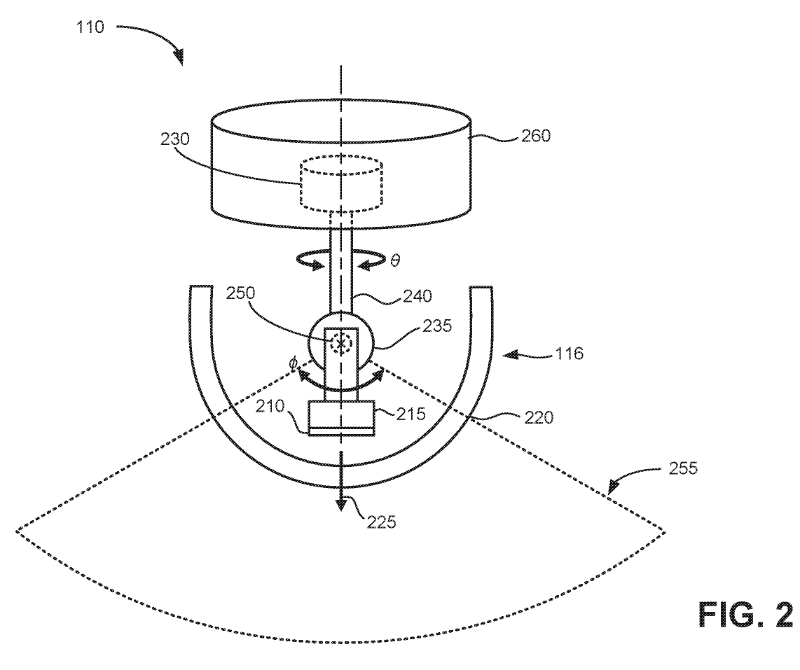

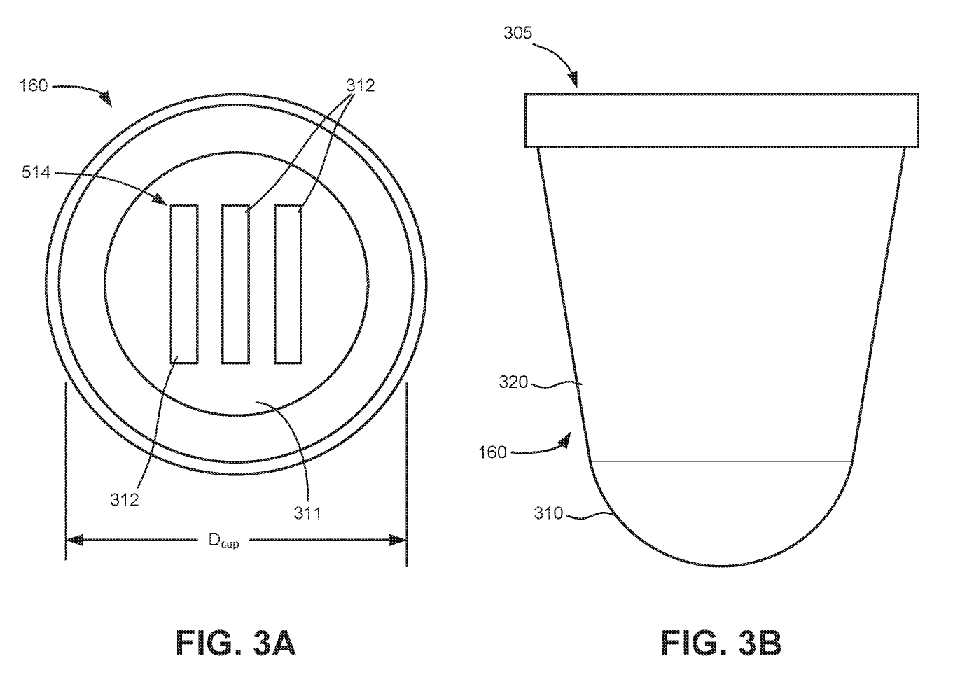

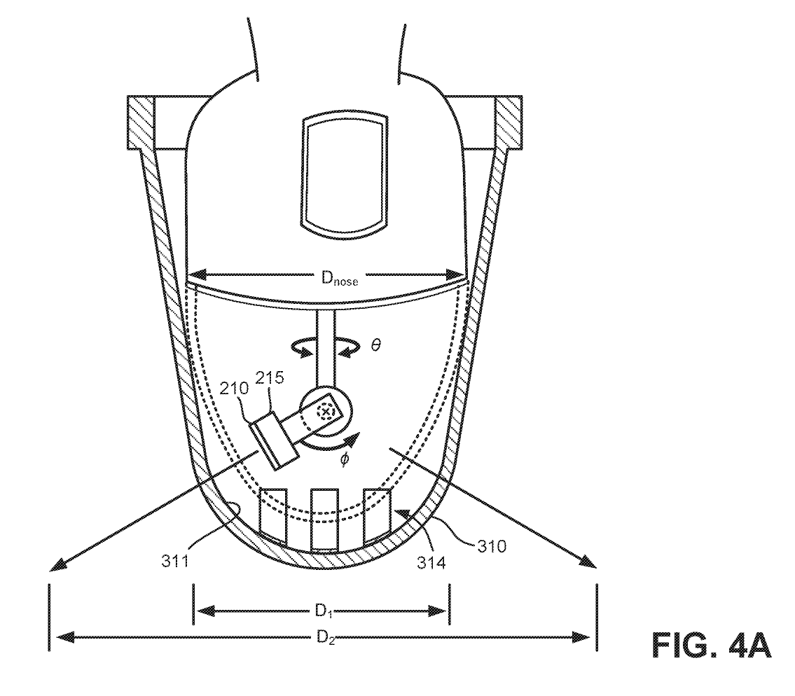

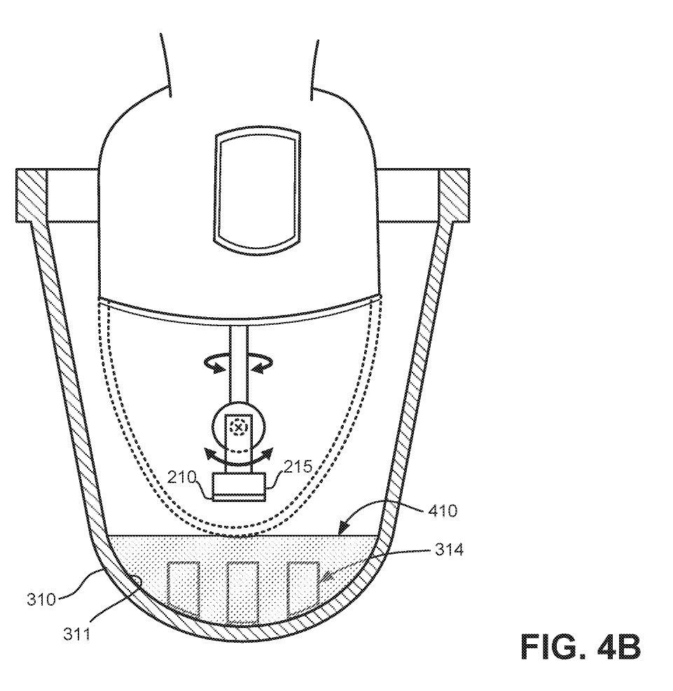

FIGS. 3A and 3B are top and side views, respectively, of calibration cup 160. FIGS. 4A and 4B are side cross-sectional views of calibration cup 160 with probe 110 inserted therein. Referring collectively to FIGS. 3A-4A, cup 160 includes an open end 305 and a closed end 310 with one or more side wall 320 in between. Open end 305 may generally be of sufficient size to accept a diameter or width of probe 110, as shown in FIG. 4A. Side wall 320 may generally provide a sufficient depth inside of cup 160 to support probe 110 when probe 110 is inserted therein through open end 305 and into contact with closed end 310.

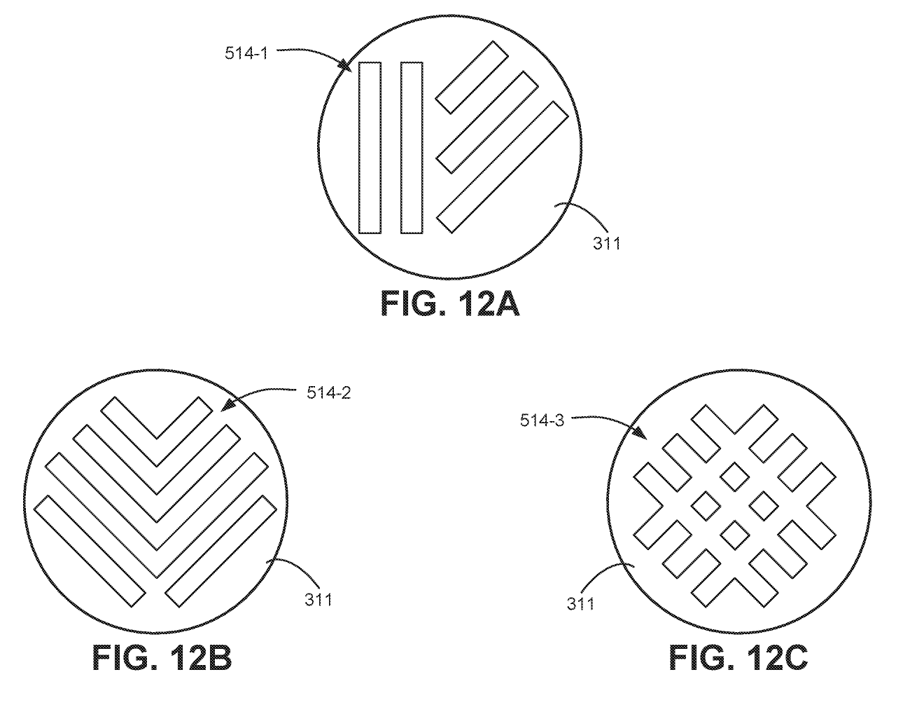

Closed end 310 may include echo-absorbing or echo-reflective structures 312 with known shapes that work together as an ultrasound target 314. Structures 312 may be secured to an inner surface 311 of closed end 310. In one implementation, inner surface 311 may include a plastic material, glass material, or another material that reflects signals differently than a testing fluid or structures 312. The arrangement of structures 312 may be any arrangement with known or predictable geometry. Thus, while structures 312 for target 314 are shown as three parallel strips in FIG. 3A, in other implementations a different arrangement of structures 312 may be used for target 314. For example, structures 312 may be arranged to include a spiral, saw tooth pattern, strips, grid, square, etc. For example, alternate arrangements for structures 512 in FIGS. 12A-12C described below may also be used with structures 312 for target 314.

According to an implementation, inner surface 311 of closed end 310 may be dome-shaped (e.g., a hollow, partly-spherical shape) to make the distance between transducer 210 and target 314 (referred to herein as the transducer-to-target distance) substantially uniform (e.g., within ten percent along a scan plane) regardless of the direction of scan line for probe 110. The uniform transducer-to-target distance allows target 314 to cover a larger scan angle with a more compact structure than conventional external calibration test fixtures. For example, as shown in FIG. 4A, the diameter, D₁, of closed end 310 with curved inner surface 311 may be less than half the diameter, D₂, required for a flat target for a 120 degree phi (ϕ) rotation of transducer 210. In other implementations, inner surface 311 may have other shapes, including flat, partly-cylindrical, or arbitrary (e.g., using flexible material). Overall, according to one implementation, the largest inner diameter (e.g., “D_{cup}” of FIG. 3A) or inner width dimension of calibration cup 160 may be no more than two times the maximum diameter of the nose portion 116 of ultrasound probe 110 (e.g., “Dₙₒₛₑ” of FIG. 4A, which may be about 4-8 cm).

Referring to FIG. 4B, in one implementation, a material 410 in which ultrasound signals can travel may be added to cup 160, when the intention is to use cup 160 as a calibration fixture. In one implementation, material 410 in cup 160 may be water or a tissue-mimicking material (e.g., a material that is comparable to human tissue in terms of speed of sound, acoustic impedance, etc.). In another implementation, a portion of cup 160 (e.g., the semi-spherical portion of closed end 310) may be provided pre-filled with another type of material 410, such as rubber, plastic, or another material for calibration purposes. When not used for calibration, the dimensions of open end 305 and wall 320 may be sufficient to support probe 110 in cup 160 with nose portion 116 resting on pre-filled material 410.

In one implementation, material 410 may include a substance in which the speed of sound travels more slowly than through water/tissue to allow for minimizing the size/diameter of inner surface 311. For example, in terms of sound travel time, a calibration cup 160 filled with rubber material 410 would be approximately a twenty-five percent smaller than a fixture using water, since the speed of sound is about twenty-five percent slower in the rubber material than in water. Thus, the overall diameter of cup 160 may be further reduced. According to one implementation, the calibration device is prefilled with a material 410 in which the speed of sound through the material is at least ten-percent slower than the speed of sound through water at room temperature (e.g., the speed of sound through the material is less than 1341 meters per second). According to another implementation, the calibration device is prefilled with a material 410 in which the speed of sound through the material is at least ten-percent slower than the speed of sound through tissue at room temperature (e.g., the speed of sound through the material is less than 1386 meters per second).

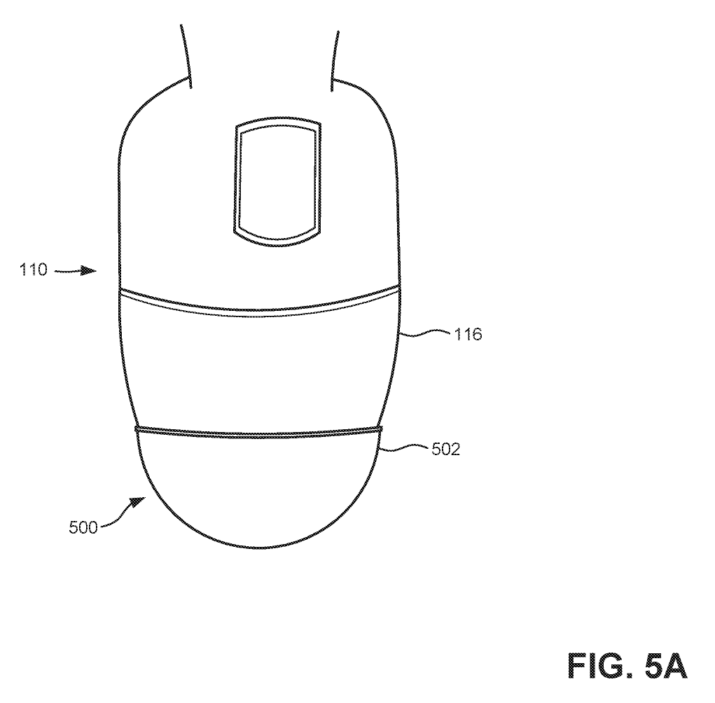

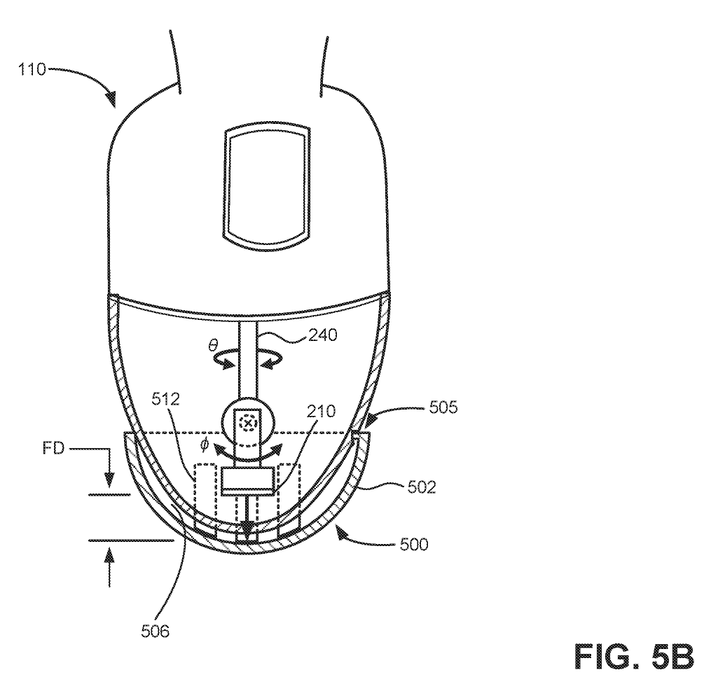

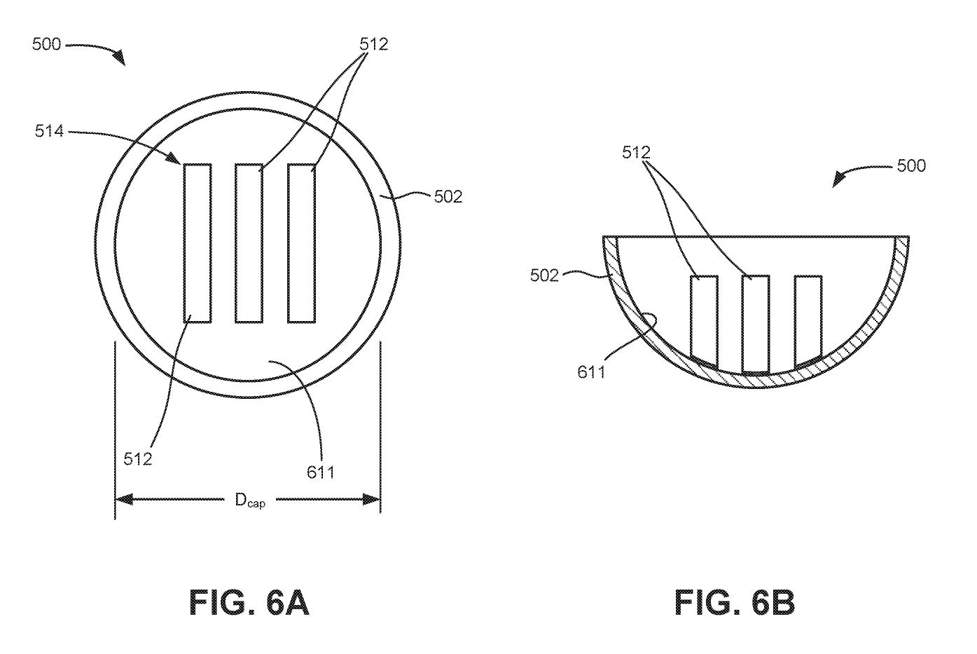

FIG. 5A is a schematic of ultrasound probe 110 with a calibration cap 500 attached, according to an implementation described herein. FIG. 5B is a partial cut-away side view of calibration cap 500 with probe 110 inserted therein. FIGS. 6A and 6B are top and side cross-sectional views, respectively, of calibration cap 500. Referring collectively to FIGS. 5A-6B, calibration cap 500 may include a supporting substrate 502 placed or secured onto an end of nose portion 116 on probe 110. In one implementation, calibration cap 500 may be held in place via an interference fit. In another implementation, calibration cap 500 and nose portion 116 may be configured with a mechanically-indexed interface 505 (e.g., uses notches) to secure calibration cap 500 to nose portion 116. In another implementation, calibration cap 500 and nose portion 116 may include indexed interface 505 to align the calibration device with the nose portion 116 without securing cap 500 to nose portion 116. In still other implementations, temporary adhesives or other securing techniques may be used. Overall, according to one implementation, the largest inner diameter (e.g., “D_{cap}” of FIG. 6A) or inner width dimension of calibration cap 500 may be less than or equal to the largest diameter of the nose portion 116 of ultrasound probe 110 (e.g., “Dₙₒₛₑ” of FIG. 4A).

Supporting substrate 502 of calibration cap 500 may include an inner surface 611 onto which structures 512 may be secured. Substrate 502 and structures 512 may be made of different materials that have different acoustic properties (acoustic impedance, attenuation, etc.). In one implementation, when calibration cap 500 is placed onto nose portion 116, structures 512 may contact the outside surface of nose portion 116 and with a gap 506 between inner surface 611 and other portions of substrate 502. Gap 506 may be air-filled, gel-filled, etc. In another implementation, both structures 512 and inner surface 611 may contact the outside surface of nose portion 116 when calibration cap 500 is placed onto nose portion 116. Thus, calibration cap 500 may form a two-substance interface on the outside surface of nose portion 116, where the two substances have different acoustic properties. Examples of interface substances include rubber structures 512 with air gaps, rubber structures 512 with gel-filled gaps 506, metal structures 512 with gel-filled gaps 506, etc.

Structures 512 may include known shapes or patterns that work together as an ultrasound target 514. In one implementation, the pattern of target 514 will show as strong shadows in air scan B-mode images. In another implementation, calibration cap 500 may be a deformable object, such as a patch or sticker, which may be applied to nose portion 116 to form a semi-spherical shape on the outside surface of nose portion 116. Target 514 may be in contact with the outer surface of nose portion 116 and/or adhere to nose portion 116. In either a rigid or deformable configuration, calibration cap 500 may be disposable or re-usable component. Thus, calibration cap 500 may be used without water, gels, or additional materials (such as any of materials 410 described in connection with FIG. 4B).

In contrast with a conventional calibration fixture, space for target 514 in calibration cap 500 is quite limited and the field distance (FD) (e.g., the transducer-to-target distance), is short (e.g., less than 10 millimeters). In some implementations, the thickness of structures 512 in calibration cap 500 may be less than the thickness of structures 312 used in calibration cup 160. In other words, when calibration cap 500 is attached to probe 110, target 514 is at the very near field where ultrasound resolution is typically not ideal. At such a short field distance, a small change in the location of target 514 could have a large impact on the resulting B-mode image. Thus, while conventional calibration methods (e.g., comparing the target shape with a ground truth shape) are still applicable, additional calibration techniques may be used to improve accuracy.

To make the thin target 514 clearly visible, calibration cap 500 utilizes ‘reverberation’ phenomenon. When probe 110 is in the air, most of the transmitted echoes are reflected back to transducer 210 at the dome-air boundary due to a very large acoustic impedance mismatch between dome 116 and air. Then, the reflected echo bounces back at the transducer 210 surface, reflects again at the dome-air boundary, hits transducer 210 again, and so on. The repeated reflections (i.e., reverberation inside dome 116) generate a horizontal stripped pattern in ultrasound images, even though there is no real target in the air. Once a calibration cap is attached to the probe, the reverberation becomes much weaker in the region where structures 512 contact dome 116 because the impedance mismatch between dome 116 and structures 512 (such as rubber material) is relatively small and the material of structures 512 used is a good echo-absorber. As a result, the scanlines that hit structures 512 show up as a dark shadow while the other scanlines that hit air still show bright reverberation patterns. This contrast between shadow and reverberation regions is very clearly visible.

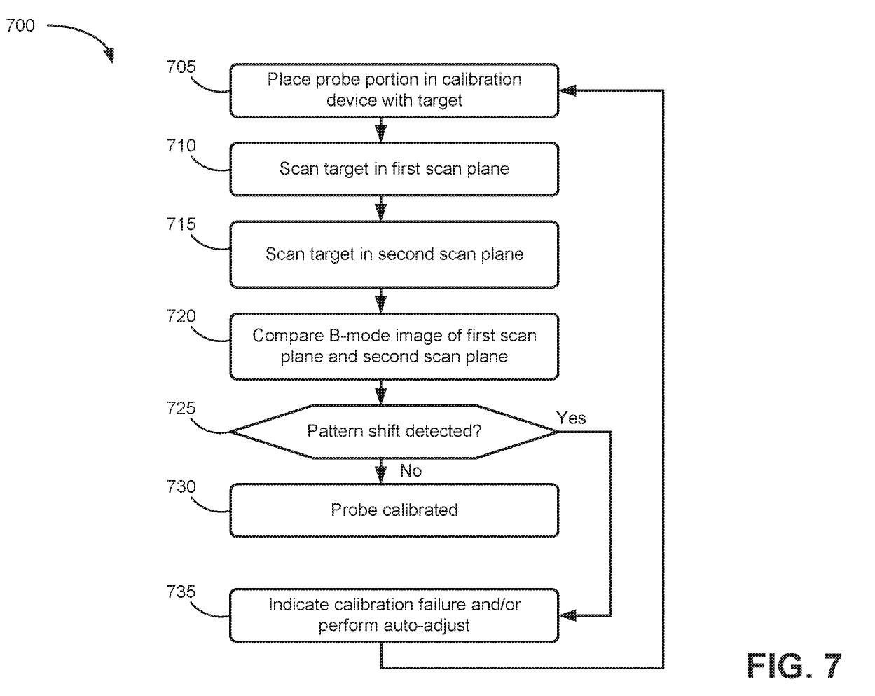

FIG. 7 is a process flow diagram of a process 700 for identifying calibration errors with compact calibration devices, according to an implementation described herein. Process 700 may include placing a portion of a probe in a calibration device having a target (block 705). For example, a technician may place probe 110 in calibration cup 160 or attach calibration cap 500 to nose portion 116.

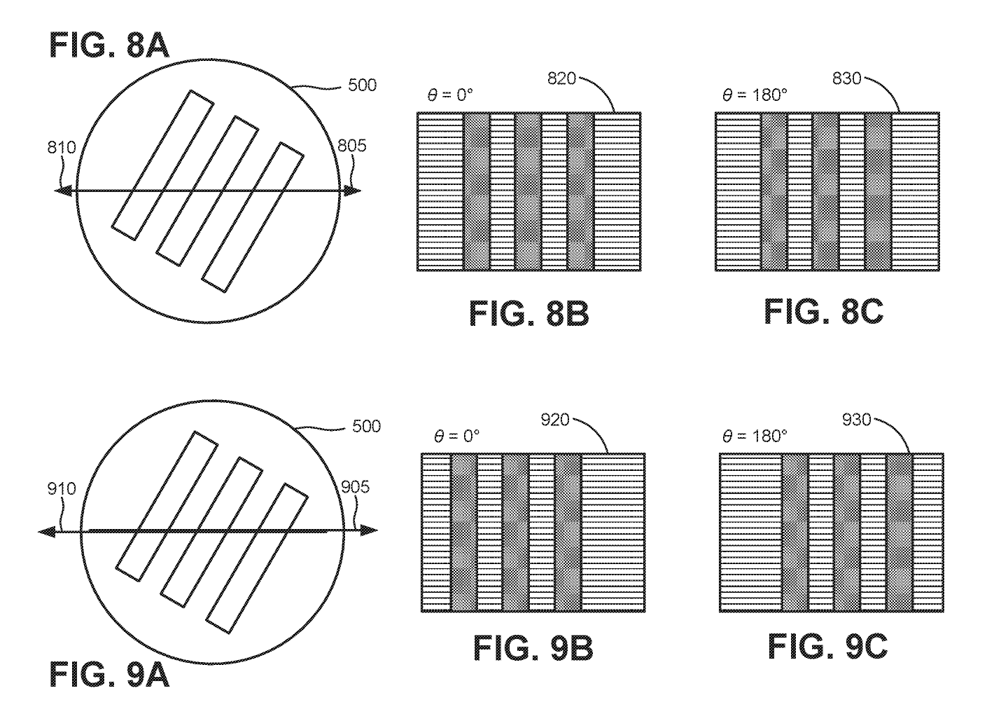

Process 700 may further include scanning the target in a first scan plane (block 710), scanning a target in a second scan plane (block 715), comparing a B-mode image from the first scan plane to a B-mode image from the second scan plane (block 720), and determining if a pattern shift is present (block 725). For example, a first scan may be performed with transducer 210 at zero degrees theta rotation and then another can performed after 180 degrees theta rotation. A comparison of the two B-mode images may reveal pattern shifts that are indicative of calibration errors. This process is described further below in connection with FIGS. 8A-10C.

If no pattern shift is detected (block 725—No), process 700 may include accepting the probe 110 as calibrated against one or more error types (block 730). For example, if the detected pattern shift is small or non-existent, no further error correction is required.

If a pattern shift is detected (block 725—Yes), process 700 may include indicating a calibration failure and/or performing an automatic adjustment (block 735). For example, base unit 120 may detect a calibration failure when a comparison of patterns from target 314 fails to conform to expected results. In one implementation, base unit 120 may indicate a calibration error. In another implementation, base unit 120 may automatically adjust the phi offset/firing delay according to the difference (e.g., fixtureless calibration). If calibration changes are needed, process 700 may be repeated to verify corrections.

FIGS. 8A-8C are simplified diagrams illustrating phi offset detection using calibration cap 500 for a probe 110 with no error, according to an implementation described herein. Transducer 210 rotates at axis 250 to scan target 314 in a first plane 805, generating a B-mode image 820. Motor 230 then rotates transducer 210 one hundred eighty degrees to scan target 314 in a second plane 810, generating another B-mode image 830. If transducer 210 is properly centered on theta axis 240, B-mode images 820 and 830 will be identical, as illustrated in FIGS. 8B and 8C.

FIGS. 9A-9C are simplified diagrams illustrating phi offset detection using calibration cap 500 for a probe 110 with a phi offset error. Transducer 210 rotates at axis 250 to scan target 314 in a first plane 905, generating a B-mode image 920. Motor 230 then rotates transducer 210 one hundred eighty degrees to scan target 314 in a second plane 910, generating another B-mode image 930. If transducer 210 is not properly centered on theta axis 240, B-mode images 920 and 930 will not be identical, as illustrated in FIGS. 9B and 9C, indicating calibration of probe 110 is required.

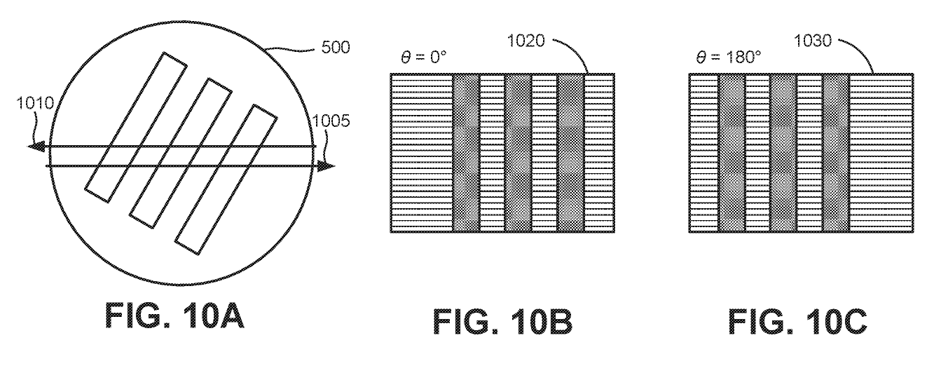

FIGS. 10A-10C are simplified diagrams illustrating perpendicular transducer skew error detection using calibration cap 500 for a probe 110. Transducer 210 rotates at axis 250 to scan target 314 in a first plane 1005, generating a B-mode image 1020. Motor 230 then rotates transducer 210 one hundred eighty degrees to scan target 314 in a second plane 1010, generating another B-mode image 1030. If transducer 210 is skewed (e.g., such that transducer 210 is not perpendicular to the intended scan plane), the planes 1005 and 1010 will not be aligned and the pattern of B-mode images 1020 and 1030 will be identical but shifted, as illustrated in FIGS. 10B and 10C, indicating calibration of probe 110 is required.

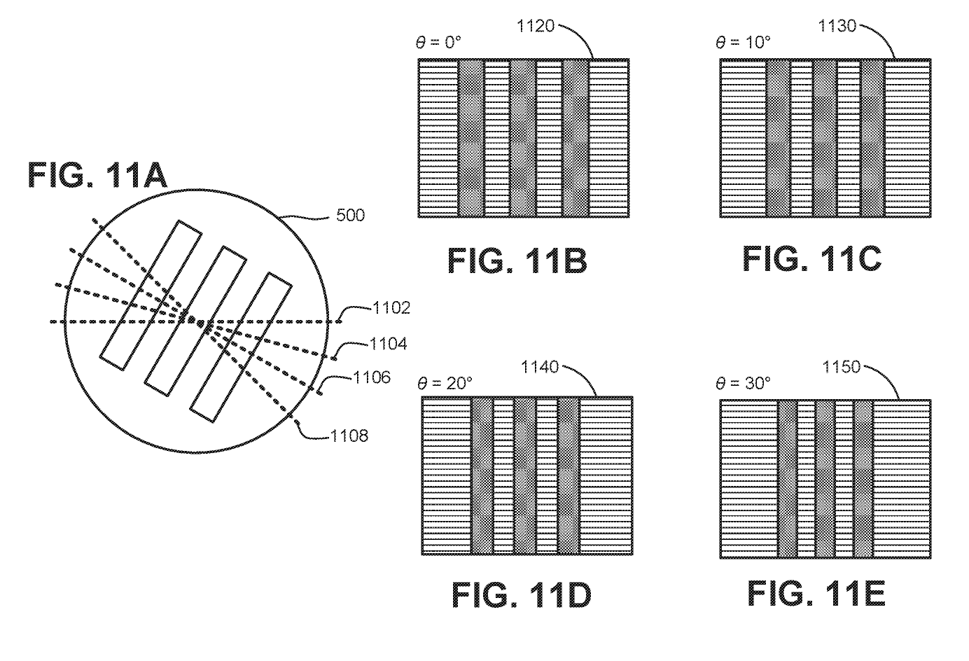

FIGS. 11A-11E are simplified diagrams illustrating theta motion error detection using calibration cap 500 for probe 110. If theta motion is correct, width/spacing of the shadow pattern changes gradually as plane number increases from 1102 to 1108. Thus, as illustrated in FIGS. 11B through 11E, the width/spacing of a target in B-mode image 1120, corresponding to scan plane 1102, would be largest. The width/spacing of the target in B-mode image 1130, corresponding to scan plane 1104, would be slightly smaller than that of B-mode image 1120. The width/spacing of the target in B-mode image 1140, corresponding to scan plane 1106, would be slightly smaller than that of B-mode image 1130. The width/spacing of the target in B-mode image 1150, corresponding to scan plane 1108, would be the smallest of the four scan planes in FIGS. 11B-11E. Detection of inconsistent B-mode images sizes and/or non-gradual width/spacing changes may be indicative of theta motion error.

FIGS. 12A-12C provide additional example patterns of targets 514. Target 514-1 in FIG. 12A includes a combination of structures 512 applied to interior surface 311 of cup 160 with different orientations. Structures 512 may be made of a material that absorbs ultrasonic energy or waves (or reflect echoes or signals differently than a testing fluid, which may include water or a tissue-mimicking material). Each of targets 514-1, 514-2, and 514-3 include structures 512 with two different angles. A v-shaped pattern (e.g., target 514-2), a grid pattern (e.g., target 514-3), a spiral pattern (not shown), or any other asymmetric pattern (e.g., target 514-1) may be used to provide more complete error information from a single scan. Particularly, multiple angles of structures 512 in a target 514 may permit distinguishing phi offset error from perpendicular error with a single scan.

In another implementation, for more accurate calibration, an indexing marker or keying mechanism may be included on nose portion 116 and calibration cap 500 to assure correct alignment of target 514 for calibration. Use of indexing may simplify use of conventional calibration approach (e.g., comparison between the ultrasound data and ground truth target shape).

Using the error detection techniques described above, several possible algorithms may be used for determining pattern shift and width/spacing estimation. In one example, a pre-processing step may be applied. A thresholding or air scan pattern detection methods, or any other pattern/texture recognition that can segment the shadow from the background, can be applied to clean up (e.g., remove noise) the ultrasound images prior to comparison. In another example, a shift estimate can be determined. Lagged cross-correlation between two images can be used to determine the amount of the shift. Also, the image phase shift can be calculated through Fourier transform. In still another example, pattern width/spacing can be estimated. An auto-correlation of an image can be used to estimate the width/spacing of the target patterns (e.g., stripe patterns of FIGS. 11B-11E). Pattern width/spacing can be estimated by detecting the dominant spatial frequency in Fourier domain. Cross/auto-correlations and Fourier transform methods are not sensitive to global pattern shifts caused by variations in probe 110/cap 500 alignment.

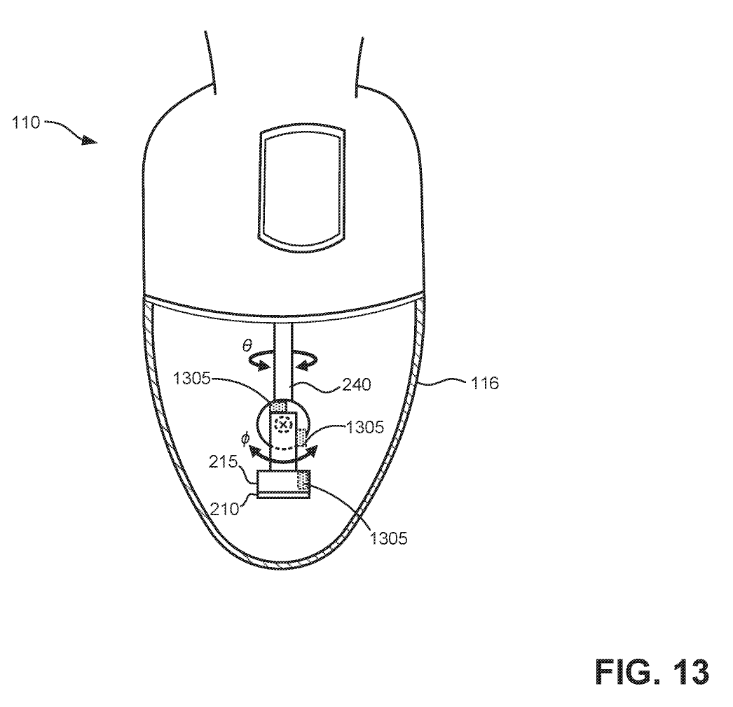

FIG. 13 is a partial cutaway view of an ultrasound probe 110 equipped for accelerometer-based calibration. As shown in FIG. 13, probe 110 may include one or more accelerometers 1305 attached to a transducer assembly (e.g., transducer bucket 215 with transducer 210).

Accelerometers 1305 may communicate with one or more transceivers located in probe 110 to communicate accelerometer data to processing components in handle 112 or base unit 120. Accelerometer 1305 may be used for calibration of probe 110. Particularly, accelerometer 1305 can detect the direction of gravity, which can be used to measure the relative angle of the transducer 210 from the gravity line. This calibration approach, using gravitational acceleration information instead of ultrasound signals, uses accelerometer 1305 mounted on a transducer bucket 215. Thus, according to an implementation, self-calibration of probe 110 may be accomplished without an external calibration fixture.

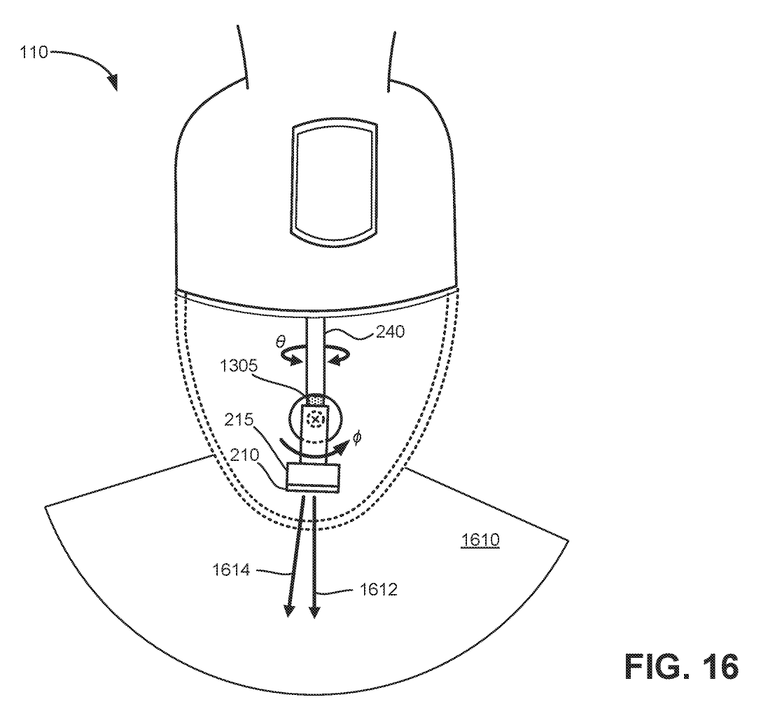

Accelerometer 1305 can detect the intensity of gravitational acceleration when accelerometer 1305 is not moving. Thus, with an accelerometer mounted on a transducer or transducer bucket, a gravity profile about the phi axis can be obtained on each scan plane. Then, the optimal phi offset can be estimated by comparing the gravity profiles before and after 180° theta rotation. As described further in connection with FIGS. 16-18, by analyzing the gravity information properly, accelerometer data can also be used for checking the integrity of phi motion and theta motion.

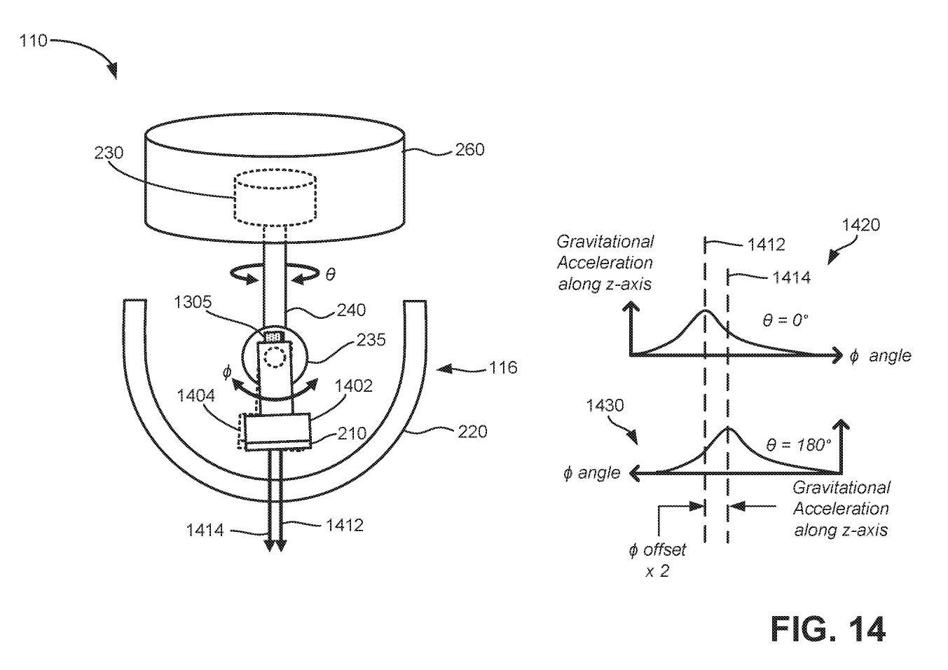

FIG. 14 is a schematic of a portion of ultrasound probe 110 with corresponding gravity intensity profiles for different transducer 210 orientations. One of the main purposes of the calibration process is to estimate appropriate phi offset and firing delay values to make the orientation of the B-mode image correct. Calibration can be done by comparing a gravity intensity profile 1420 of transducer 210 in one scan plane (e.g., position 1402) with another gravity intensity profile 1430 of transducer 210 in the same scan plane after 180° theta rotation (e.g., position 1404) as shown in FIG. 14. For example, for a system that does not have any phi error, the maximum peak intensity angles in the first and second profiles, ϕpeak1 (ref. line 1412) and ϕpeak2 (ref. line 1414), respectively, would have the relationship, ϕpeak1=180°−ϕpeak2. For a system where the actual phi angle of the transducer 210 is skewed by ϕoffset from the correct direction, two angles of maximum peak would be determined by the following equation:

2*ϕoffset=ϕpeak1−(180°−ϕpeak2).

By utilizing this relationship between peak intensity angles, the phi offset can be calibrated.

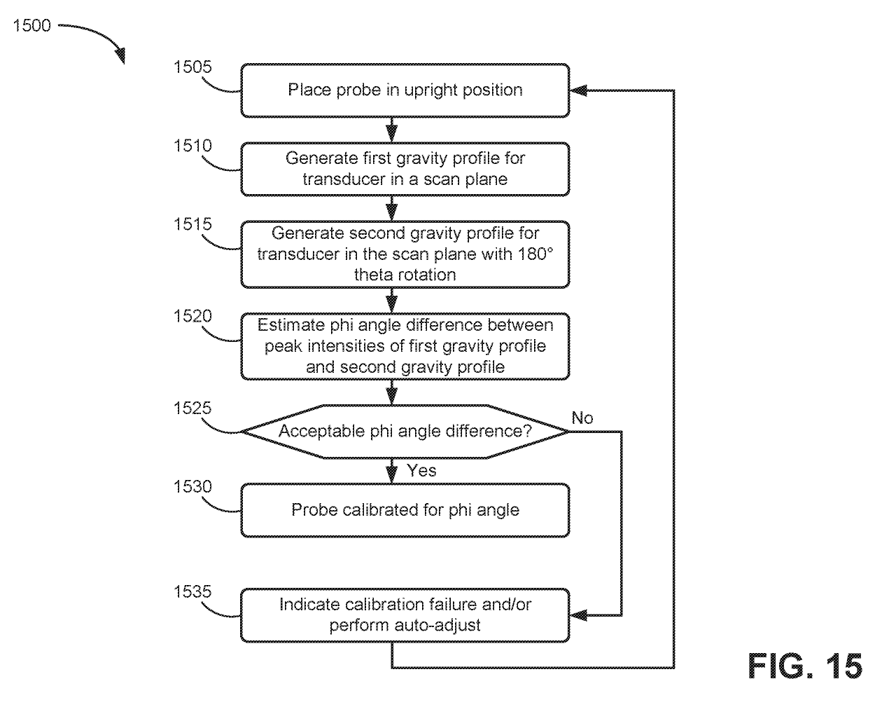

FIG. 15 is a process flow diagram of a process 1500 for estimating phi angle probe error using accelerometer data, according to an implementation described herein. Process 1500 may include placing a probe in an upright position (block 1505). For example, a technician may fix probe 110 in a generally upright position. Probe 110 placement need not be perfectly aligned with vertical. An upright probe position is recommended for strong gravitational acceleration signal intensity, but the probe position does not have to be perfectly upright. A typical probe holder on a cart (e.g., cart 140) would provide a good position for holding probe 110. If probe 110 has a flat top or base, standing probe 110 on its top or base could be another convenient approach.

Process 1500 may further include generating a first gravity profile for the transducer in a first scan plane (block 1510), and generating a second gravity profile for the transducer in the same scan plane with 180° theta rotation (block 1515). For example, using accelerometer 1305, the intensities of gravitational acceleration may be measured along the beam directions in a scan plane to generate a gravity profile. Transducer/transducer bucket 210/215 motion highly affects the accelerometer 1305 reading. Thus, it should be ensured that theta motor 230 and phi motor 235 do not move during accelerometer measurement (e.g., by slowing down each stepping motion). Another similar measurement may be performed after 180° theta rotation and another gravity profile generated in the same scan plane with 180° rotation.

Process 1500 may also include estimating a phi angle difference between peak intensities of the first gravity profile and the second gravity profile (block 1520). For example, the phi angle difference between the two profiles may be estimated using graphs 1420 and 1430 shown in in FIG. 14. A peak detection or cross correlation method can be used after flipping one of the gravity profiles. Accelerometer 1305 mounted on transducer bucket 215 could have some skewed angle; i.e., accelerometer direction could be tilted from real ultrasound beam direction due to an assembly error. In addition, different motor speeds between real exam scan and gravity measurement could add more offset angle due to the magnetic spring effect at the motor shaft. This offset angle can be measured once during manufacturing after an ultrasound-based factory calibration.

Process 1500 may further include determining if the phi angle difference is acceptable (block 1525). For example, base unit 120 may determine if the estimated phi angle difference is below a set threshold for acceptable probe performance.

If the phi angle difference is acceptable (block 1525—Yes), process 1500 may include accepting the probe as calibrated for the phi angle (block 1530). For example, if the estimated phi angle difference is small or non-existent, the current firing delay values (e.g., specific delay times for firing each group of elements in order to generate the desired beam shape) and/or phi offset values may be used for calibration.

If the phi angle difference is not acceptable (block 1525—No), process 1500 may include indicating a calibration failure and/or performing an automatic adjustment (block 1535). For example, base unit 120 may detect a calibration failure (fault detection) when the estimated phi angle difference exceeds the threshold value. In one implementation, base unit 120 may indicate a calibration error. In another implementation, base unit 120 may automatically adjust the phi offset/firing delay according to the difference (e.g., fixtureless calibration). If calibration changes are needed, process 1500 may be repeated to verify corrections.

FIG. 16 is a schematic of a portion of ultrasound probe 110 showing vectors used for a phi motion integrity check. As shown in FIG. 16, a gravitational force vector 1612 and a center scanline 1614 are in the same scan plane 1610. As used herein, a “center scanline” may generally refer to a vector that cuts the B-mode sector in half. Theoretically, scanlines at the exact center of each B-mode sector should correspond to center scanline 1614 if there is no assembly error. If probe 110 is positioned perfectly vertical, center scanline 1614 should match the direction of gravitational force vector 1612. In a scan plane that is perpendicular to the gravitational force, theoretical values of measured gravity can be calculated using the following equation:

g_{measured}(ϕ_{g})=g_{(standard gravity)}×cos(ϕ)_{g})

where ϕ_{g} is the angle between a scanline and the direction of gravity.

When probe 110 orientation is close to vertical, at least one scan plane (e.g., scan plane 1610) should contain both of gravitational force vector 1612 and broadside vector 1614, as depicted in FIG. 16. This assumption can be reasonably made when the number of scan planes by probe 110 is large, such as 24 or more. In this perpendicular plane, the direction of gravity corresponds to the scanline with the maximum acceleration value from accelerometer 1305. Then, a theoretical gravity profile, a sinusoidal-shaped curve symmetrical about the maximum acceleration angle, can be calculated using the abovementioned equation. If errors between the theoretical values and actual measurements are large, it implies that phi motion is not correct.

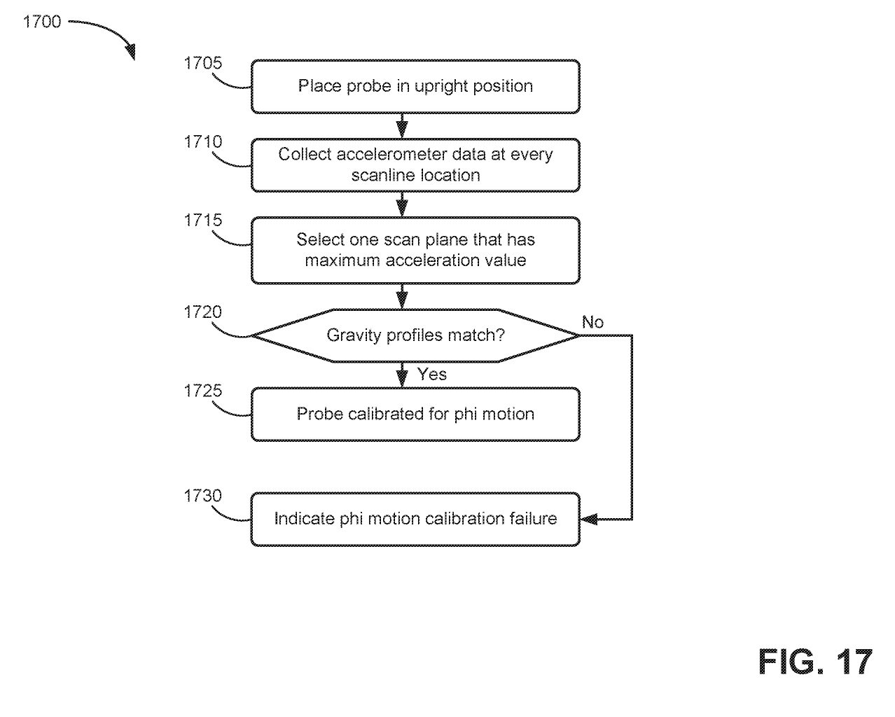

FIG. 17 is a process flow diagram of a process 1700 for detecting phi motion probe error using accelerometer data, according to an implementation described herein. In one implementation, process 1700 may be conducted after performing the phi offset calibration process of FIG. 15.

Process 1700 may include placing a probe in an upright position (block 1705). For example, a technician may fix probe 110 in a generally upright position. Probe 110 placement need not be perfectly aligned with vertical. An upright probe position is recommended for strong gravitational acceleration signal intensity, but the probe position does not have to be perfectly vertical or upright.

Process 1700 may also include collecting accelerometer data at every scanline location (block 1710) and selecting one plane that has a maximum acceleration value (block 1715). For example, for each scan plane (each available 0 angle) of probe 110, accelerometer 1305 readings may be collected along the transducer 210 phi motion range. A plane with the highest acceleration value (e.g., plane 1610), which would be parallel to the direction of gravity, may be selected.

Process 1700 may further include determining if the measured gravity profile in the selected plane matches a theoretical gravity profile (block 1720). For example, base unit 120 may generate a gravity profile based on the measured accelerometer data and another gravity profile based on the theoretical position data of transducer 210.

If differences in the gravity profiles are minimal (block 1720—Yes), process 1700 may include accepting the probe as calibrated for the phi motion (block 1725). For example, if differences in the measured and theoretical gravity profiles are small, probe 110 may be accepted for phi motion calibration.

If the differences in the gravity profiles are not acceptable (block 1720—No), process 1700 may include indicating a calibration failure (block 1730). For example, base unit 120 may detect a calibration failure (fault detection) when the differences in the gravity profiles exceed a threshold value. In one implementation, base unit 120 may indicate a calibration error.

Referring again to FIG. 14, when probe 110 is in an upright position, changes to the theta angle do not affect accelerometer 1305 readings because the theta rotation axis 240 is parallel to the direction of gravity. To check the theta motion integrity, probe 110 can be tilted to a horizontal position (e.g., laying probe 110 on its side) to make accelerometer 1305 sensitive to theta angle changes. In horizontal probe position, the two gravity profiles measured at the first scan plane location before and after 180° theta rotation should match each other after flipping the second one, if there is no theta motor problem. The only exception is when the first scan plane is horizontal making the gravity profile perfectly symmetric for all phi angle positions (i.e., “no theta motion” is not distinguishable from “180° theta rotation”). To avoid this problem and to make the gravity profile as asymmetric as possible, probe 110 may be rotated to make the first scan plane close to vertical.

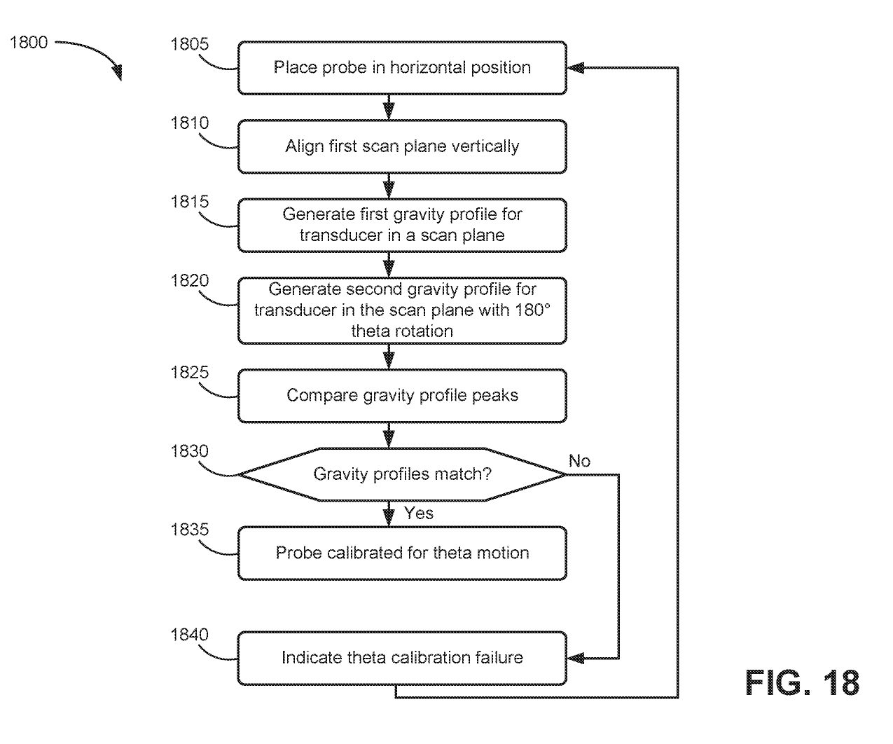

FIG. 18 is a process flow diagram of a process 1800 for estimating theta motion error in probe 110 using accelerometer data, according to an implementation described herein. Process 1800 may include placing a probe in a horizontal position (block 1805) and aligning a first scan plane vertically (block 1810). For example, a technician may lay probe 110 on its side and hold probe 110 in place to avoid rolling/rotating. Probe 110 placement need not be perfectly horizontal. Probe 110 may be positioned so that the first scan plane (e.g., corresponding to motion of phi motor 235) is generally vertical.

Process 1800 may further include generating a first gravity profile for the transducer in a first scan plane (block 1815), and generating a second gravity profile for the transducer in the same scan plane with 180° theta rotation (block 1820). For example, using accelerometer 1305, the intensities of gravitational acceleration may be measured along the beam directions in a scan plane to generate a gravity profile. Transducer/transducer bucket motion highly affects the accelerometer 1305 reading. Thus, it should be ensured that theta motor 230 and phi motor 235 are stationary during accelerometer measurement reading (e.g., by slowing down each stepping motion). Another similar measurement may be performed after 180° theta rotation and another gravity profile generated in the same scan plane with 180° rotation.

Process 1800 may also include comparing peak intensities of the first gravity profile and the second gravity profile (block 1825). For example, the theta motion difference between the two profiles may be estimated using graphs similar to those shown in in FIG. 14. A peak detection or cross correlation method can be used after flipping (e.g., along the gravitational acceleration axis, as in graph 1430) one of the gravity profiles.

Process 1800 may further include determining if the gravity profiles match (block 1830). For example, base unit 120 may determine if the estimated peak location difference between the first gravity profile and the second gravity profile is below a threshold for acceptable probe performance.

If the gravity profiles match (block 1830—Yes), process 1800 may include accepting the probe as calibrated for the theta motion (block 1835). For example, if the estimated peak offset is small or non-existent, probe 110 may be deemed calibrated for theta motion.

If the theta motion difference is not acceptable (block 1830—No), process 1800 may include indicating a calibration failure and/or performing an automatic adjustment (block 1840). For example, base unit 120 may detect a calibration failure when the estimated theta angle difference exceeds the threshold value. In one implementation, base unit 120 may indicate a calibration error. In another implementation, base unit 120 may automatically adjust the theta offset according to the difference (e.g., fixtureless calibration). If calibration changes are performed, process 1800 may be repeated to verify corrections.

According to another implementation, accelerometer 1305 may include a three-axis accelerometer attached to a transducer assembly of probe 110. As a three-axis accelerometer, accelerometer 1305 can measure the magnitude and direction of acceleration in three-dimensional space. If a three-axis accelerometer 1305 is mounted on transducer 210 or transducer bucket 215 (e.g., as shown in FIG. 13), the relative angle between any two scanlines can be measured.

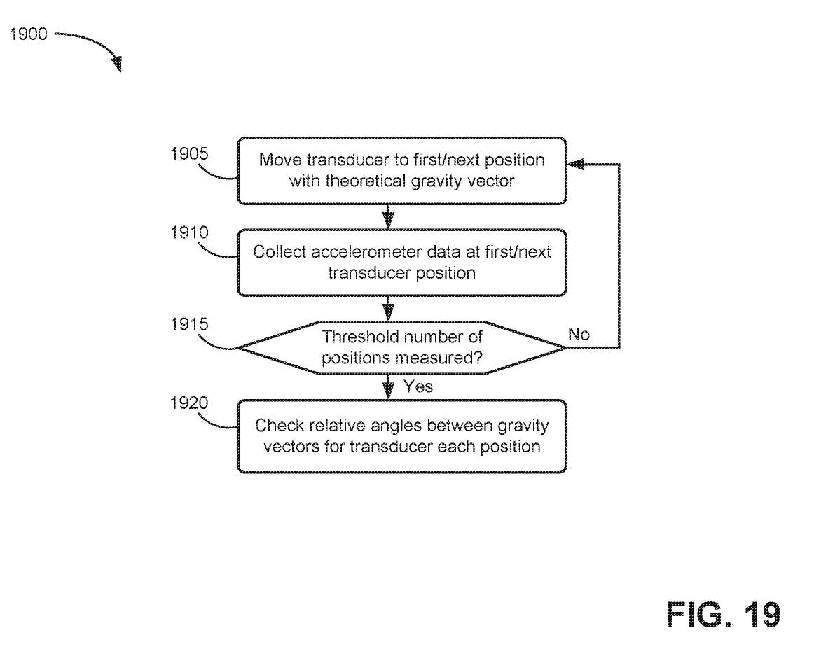

FIG. 19 is a process flow diagram of a process 1900 for estimating errors in probe 110 using gravity angle information from a three-axis accelerometer, according to an implementation described herein.

Process 1900 may include moving the transducer to a first position (block 1905) and measuring the three-dimensional direction of a gravity vector at the position (block 1910). For example, a technician may hold probe 110 in place and cause transducer 210/transducer bucket 215 to move to a first position (e.g., a particular phi angle and theta angle). The three-dimensional direction of a gravity vector at the first position may be obtained using three-axis accelerometer 1305.

Process 1900 may include determining if a threshold number of positions have been measured (block 1915). For example, in one implementation, at least two position measurements may be required to perform a comparison. If a threshold number of positions have not been measured (block 1915—No), process 1900 may include repeating the moving and measure steps for another transducer position (block 1915). For example, the transducer may be moved to another position that should have a gravity vector in the same direction.

If a threshold number of positions have been measured (block 1915—Yes), process 1900 may include checking the relative angles between the gravity vectors match for each transducer position (block 1920). For example, once a threshold number of transducer positions have been measured, the measured gravity vector for each position may be compared. In one implementation the integrity of phi motion may be determined by comparing the gravity angles before and after a phi motion. In another implementation, the integrity of theta motion may be checked by comparing the gravity angles before and after a theta motion. In still another implementation, the phi offset angle may be checked by comparing the gravity angles before and after 180-degree theta motion. For example, the gravity angles at two positions: (phi=−45°, theta=0°) and (phi=+45°, theta=180°) could be compared. If the gravity angles at each position do not match each other, it means the phi offset is wrong. Probe 110 may try several different phi offsets until the two match to determine the correct phi offset.

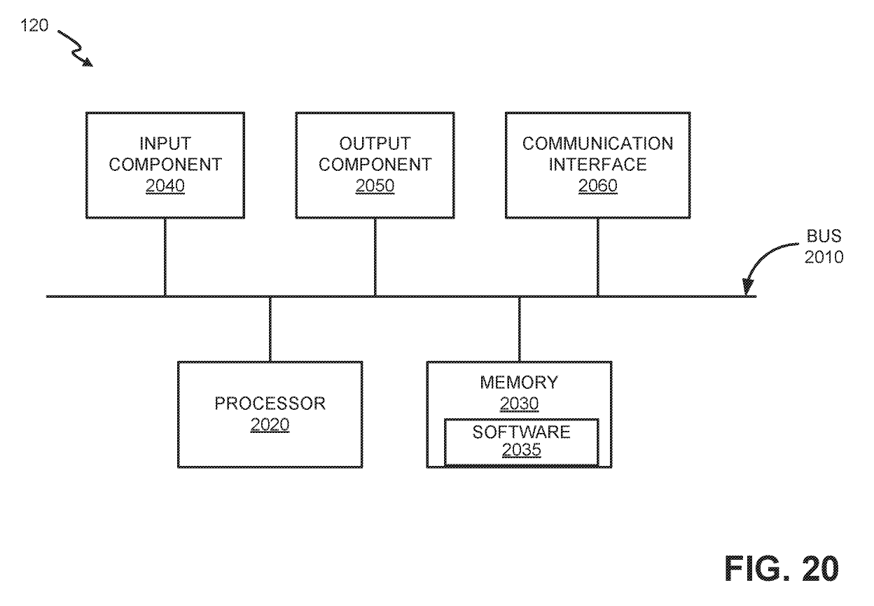

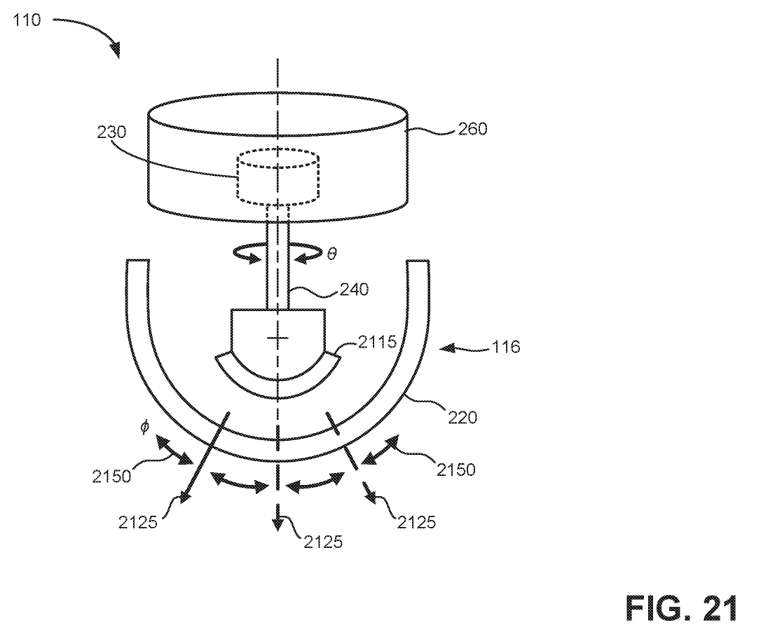

FIG. 20 is a block diagram illustrating exemplary physical components of base unit 120. Additionally, or alternatively, probe 110 may include similar components. Base unit 120 may include a bus 2010, a processor 2020, a memory 2030, an input component 2040, an output component 2050, and a communication interface 2060.

Bus 2010 may include a path that permits communication among the components of base unit 120. Processor 2020 may include a processor, a microprocessor, or processing logic that may interpret and execute instructions. Memory 2030 may include any type of dynamic storage device that may store information and instructions (e.g., software 2035), for execution by processor 2020, and/or any type of non-volatile storage device that may store information for use by processor 2020.

Software 2035 includes an application or a program that provides a function and/or a process. Software 2035 is also intended to include firmware, middleware, microcode, hardware description language (HDL), and/or other form of instruction.

Input component 2040 may include a mechanism that permits a user to input information to base unit 120, such as a keyboard, a keypad, a button, a switch, a touch screen, etc. Output component 2050 may include a mechanism that outputs information to the user, such as a display (e.g., an LCD), a speaker, one or more light emitting diodes (LEDs), etc.

Communication interface 2060 may include a transceiver that enables base unit 120 to communicate with other devices and/or systems via wireless communications, wired communications, or a combination of wireless and wired communications. For example, communication interface 2060 may include mechanisms for communicating with another device or system, such as probe 110, via a network, or to other devices/systems, such as a system control computer that monitors operation of multiple base units (e.g., in a hospital or another type of medical monitoring facility). In one implementation, communication interface 2060 may be a logical component that includes input and output ports, input and output systems, and/or other input and output components that facilitate the transmission of data to/from other devices.

Base unit 120 may perform certain operations in response to processor 2020 executing software instructions (e.g., software 2035) contained in a computer-readable medium, such as memory 2030. A computer-readable medium may be defined as a non-transitory memory device. A non-transitory memory device may include memory space within a single physical memory device or spread across multiple physical memory devices. The software instructions may be read into memory 2030 from another computer-readable medium or from another device. The software instructions contained in memory 2030 may cause processor 2020 to perform processes described herein. Alternatively, hardwired circuitry, such as an application-specific integrated circuit (ASIC), a field-programmable gate array (FPGA), etc., may be used in place of or in combination with software instructions to implement processes described herein. Thus, implementations described herein are not limited to any specific combination of hardware circuitry and software.

Base unit 120 may include fewer components, additional components, different components, and/or differently arranged components than those illustrated in FIG. 20. As an example, base unit 120 may include one or more switch fabrics instead of, or in addition to, bus 2010. Additionally, or alternatively, one or more components of base unit 120 may perform one or more tasks described as being performed by one or more other components of base unit 120.

Systems and methods described herein allow for compact calibration of 3D ultrasound probes. In one embodiment, a calibration device for an ultrasound probe has an open end to receive a nose portion of the ultrasound probe; a closed end including a bottom inner surface having hollow partly-spherical shape; and a target secured to the bottom inner surface, the target comprising an echo- or signal-absorbing material.

In another embodiment, a method for calibrating an ultrasound probe is provided. The ultrasound probe includes a transducer assembly configured to rotate about a theta axis and a phi axis. The method includes inserting a nose portion of the probe into a calibration device. The calibration device includes an open end to receive a nose portion of the ultrasound probe, a closed end including a bottom inner surface having a hollow partly-spherical shape, and a target of echo- or signal-absorbing material secured to the bottom inner surface. The method also includes scanning the target in a first scan plane at a first theta angle to generate a first B-mode image; scanning the target in a second scan plane at a second theta angle to generate a second B-mode image; and comparing the first B-mode image with the second B-mode image to identify a pattern shift of the target between the first B-mode image and the second B-mode image.

In still another embodiment, a system includes an ultrasound probe and a base unit. The ultrasound probe includes a transducer assembly configured to rotate about a theta axis and a phi axis, and an accelerometer mounted on the transducer assembly. The base unit is configured to receive, from the accelerometer, first accelerometer data for a first scan plane corresponding to a first theta angle; receive, from the accelerometer, second accelerometer data for a second scan plane corresponding to a second theta angle, the second theta angle being 180 degrees from the first theta angle; generate a first gravity profile for the first scan plane and a second gravity profile for the second scan plane; and estimate, based on a comparison of the first gravity profile and the second gravity profile, a phi angle difference between the first theta angle and the second theta angle.

The foregoing description of exemplary implementations provides illustration and description, but is not intended to be exhaustive or to limit the embodiments described herein to the precise form disclosed. Modifications and variations are possible in light of the above teachings or may be acquired from practice of the embodiments.

Although the invention has been described in detail above, it is expressly understood that it will be apparent to persons skilled in the relevant art that the invention may be modified without departing from the spirit of the invention. Various changes of form, design, or arrangement may be made to the invention without departing from the spirit and scope of the invention.

For example, FIG. 21 is a schematic of a portion of probe 110 according to another implementation. In the configuration of FIG. 21, probe 110 includes an array transducer 2115. Array transducer 2115 may include a curved array (e.g., as shown in FIG. 21) or a linear array. Array transducer 2115 may provide an ultrasonic beam 2125 that may move in the phi direction 2150 without a motor (e.g., without motor 235 of FIG. 2) Similar to motor 235, array transducer 2115 may be mounted for theta rotation around axis 240. Thus, similar calibration procedures to those described above may be used for the configuration of probe 110 in FIG. 21, such as pattern shift detection and comparison of gravity profiles.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

Use of ordinal terms such as “first,” “second,” “third,” etc., in the claims to modify a claim element does not by itself connote any priority, precedence, or order of one claim element over another, the temporal order in which acts of a method are performed, the temporal order in which instructions executed by a device are performed, etc., but are used merely as labels to distinguish one claim element having a certain name from another element having a same name (but for use of the ordinal term) to distinguish the claim elements.

What is claimed is:

1. A calibration device for an ultrasound probe, comprising:
an open end to receive a nose portion of the ultrasound probe;
a closed end including an inner surface; and
a target secured to the inner surface, the target comprising one of an echo-absorbing material or an echo-reflecting material, wherein the calibration device has an inner width dimension that is no more than two times the maximum nose diameter of the ultrasound probe.

2. The calibration device of claim 1, further comprising:
a side wall between the open end and the closed end, the side wall forming a depth in the calibration device to support the ultrasound probe in an upright position when the ultrasound probe is inserted through the open end and the nose portion is in contact with the closed end.

3. The calibration device of claim 1, wherein the calibration device is configured to receive water or an ultrasound gel for use during a calibration procedure.

4. The calibration device of claim 1, wherein the calibration device is pre-filled with a solid material.

5. The calibration device of claim 1, wherein the calibration device is pre-filled with a material in which the speed of sound through the material is at least ten-percent slower than the speed of sound through water.

6. The calibration device of claim 1, further comprising:
an attachment mechanism for securing the calibration device to a cart.

7. The calibration device of claim 1, further comprising:
an attachment mechanism for removeably attaching the calibration device to the nose portion.

8. The calibration device of claim 1, wherein the ultrasound probe includes a single element transducer that rotates about two different axes or an annular array transducer.

9. The calibration device of claim 8, wherein the target secured to the inner surface covers a probe scan angle of at least 120 degrees about a phi axis that is orthogonal to a longitudinal axis of the ultrasound probe.

10. The calibration device of claim 1, wherein the ultrasound probe includes an array transducer.

11. The calibration device of claim 1, wherein the calibration device provides a substantially uniform transducer-to-target distance.

12. The calibration device of claim 1, wherein the target contacts the nose portion during a calibration procedure.

13. The calibration device of claim 1, wherein the calibration device is deformable and adheres to an outside surface of the nose portion.

14. The calibration device of claim 1, wherein the calibration device includes an indexed portion to align the calibration device with the nose portion.

15. A method for calibrating an ultrasound probe, the ultrasound probe including a transducer assembly configured to rotate about a theta axis and a phi axis, the method comprising:
inserting a nose portion of the probe into a calibration device, the calibration device including:
an open end to receive a nose portion of the ultrasound probe,
a closed end including an inner surface, and
a target secured to the inner surface;
scanning the target in a first scan plane at a first theta angle to generate a first ultrasound image;
scanning the target in a second scan plane at a second theta angle to generate a second ultrasound image; and
comparing the first ultrasound image with the second ultrasound image to identify a pattern shift of the target between the first ultrasound image and the second ultrasound image.

16. The method of claim 15, wherein the first theta angle and the second theta angle are 180 degrees apart.

17. The method of claim 15, wherein the target includes two or more parallel strips.

18. The method of claim 15, further comprising:
attaching the calibration device to the nose portion.

19. The method of claim 15, further comprising:
adding water or a ultrasound gel in the calibration device.

20. The method of claim 15, wherein the calibration device includes a solid material between the ultrasound probe and the target.

21. A system comprising:
an ultrasound probe, including:
a transducer assembly configured to rotate about a theta axis and a phi axis, and
an accelerometer mounted on the transducer assembly; and
a processing unit configured to:
move the transducer assembly to a first position with a first theta angle,
receive, from the accelerometer, first accelerometer data corresponding to a first theta angle of the transducer assembly,
move the transducer assembly to a second position with a second theta angle,
receive, from the accelerometer, second accelerometer data corresponding to a second theta angle of the transducer assembly, the second theta angle being different from the first theta angle,
generate a first gravity profile for the first theta angle and a second gravity profile for the second theta angle, and
estimate, based on a comparison of the first gravity profile and the second gravity profile, a calibration error between the transducer assembly at the first theta angle and the transducer assembly at the second theta angle.

22. The system of claim 21, wherein the accelerometer includes a three-axis accelerometer.
